(19) United States
(12) Patent Application Publication
(10) Pub. No.: US 2025/0281085 A1
(43) Pub. Date: Sep. 11, 2025

(54) SYSTEMS AND METHODS FOR PERFORMING A MOTOR SKILLS NEUROLOGICAL TEST USING AUGMENTED OR VIRTUAL REALITY

(71) Applicant: Magic Leap, Inc., Plantation, FL (US)

(72) Inventors: Mattia DEMASI, Chicago, IL (US); Edward NYMAN, JR., Maumee, OH (US); Emilio Patrick SHIRONOSHITA, Miami, FL (US)

(73) Assignee: Magic Leap, Inc., Plantation, FL (US)

(21) Appl. No.: 18/854,496

(22) PCT Filed: Apr. 8, 2022
(86) PCT No.: PCT/US2022/024075

§ 371 Date: Oct. 4, 2024

(51) Int. Cl. A61B 5/16; A61B 5/00; A61B 5/11; G06F 3/01

(52) U.S. Cl. CPC A61B 5/163; A61B 5/1114; A61B 5/4082; G06F 3/013

(57) ABSTRACT

System and methods for performing a motor skills neurological test using augmented reality which provides an objective assessment of the results of the test. A virtual target is displayed to a user in an AR field of view of an AR system at a target location. The movement of a body part (e.g., a finger) of a user is tracked as the user moves the body part from a starting location to the target location. A total traveled distance of the body part in moving from the starting location to the target location is determined based on the tracking. A linear distance between the starting location and the target location is determined. An efficiency index is then determined which represents an overall quality of movement of the body part from the starting location to the target location based on the total traveled distance and the linear distance.

### FIELD OF THE INVENTION

The present disclosure relates to virtual reality systems, and more particularly, to systems and methods for performing and assessing/quantifying a motor skills neurological test using virtual reality.

### BACKGROUND

Modern computing and display technologies have facilitated the development of systems for so called “virtual reality” (VR), “augmented reality” (AR), and/or “mixed-reality” (MR) experiences, wherein digitally reproduced images, or portions thereof, are presented to a user in a manner wherein they seem to be, or may be perceived as, real. A VR scenario typically involves presentation of digital or virtual image information without transparency to actual real-world visual input. An AR scenario typically involves presentation of digital or virtual image information as an augmentation to visualization of the real-world around the user (i.e., transparency to real-world visual input). Accordingly, AR scenarios involve presentation of digital or virtual image information with transparency to the real-world around the user. An MR scenario is a version of an AR scenario, except with more extensive merging of the real world and virtual world in which physical objects in the real world and virtual objects may co-exist and interact in real-time. As used herein, the terms “extended reality” an (“XR”) is used to refer collectively to any of VR, AR and/or MR. In addition, the term AR means either, or both, AR and MR.

Various optical systems generate images at various depths for displaying VR scenarios. Some such optical systems are described in U.S. Utility patent application Ser. No. 14/555,585 filed on Nov. 27, 2014 (attorney docket number ML.20011.00), the contents of which are hereby expressly and fully incorporated by reference in their entirety, as though set forth in full.

XR systems typically employ wearable display devices (e.g., head-worn displays, helmet-mounted displays, or smart glasses) that are at least loosely coupled to a user's head, and thus move when the user's head moves. If the user's head motions are detected by the display device, the data being displayed can be updated to take the change in head pose (i.e., the orientation and/or location of user's head) into account.

As an example, if a user wearing a head-worn display device views a virtual representation of a virtual object on the display device and walks around an area where the virtual object appears, the virtual object can be rendered for each viewpoint (corresponding to a position and/or orientation of the head-worn display device), giving the user the perception that they are walking around an object that occupies real space. If the head-worn display device is used to present multiple virtual objects at different depths, measurements of head pose can be used to render the scene to match the user's dynamically changing head pose and provide an increased sense of immersion. However, there is an inevitable lag between rendering a scene and displaying/projecting the rendered scene.

Head-worn display devices that enable AR provide concurrent viewing of both real and virtual objects. With an “optical see-through” display, a user can see through transparent (or semi-transparent) elements in a display system to directly view the light from real objects in a real-world environment. The transparent element, often referred to as a “combiner,” superimposes light from the display over the user's view of the real world, where light from the display projects an image of virtual content over the see-through view of the real objects in the environment. A camera may be mounted onto the head-worn display device to capture images or videos of the scene being viewed by the user.

Clinically deployed neurological assessments often include variants of the finger-to-nose tracking test (FNT). In such tests, the patient is tasked with moving a finger (e.g., the index finger) of one hand from a starting point to the patient's nose while a trained healthcare provider (e.g., a clinician such as a physician, neurologist, or the like) observes the patient's performance. Commonly, such tests are currently evaluated subjectively, albeit by the trained clinician, wherein movement characteristics are qualitatively assessed by the trained clinician. The results of the test inform health conditions such as assessments of dysmetria (a lack of coordination of movement typified by the undershoot or overshoot of intended position with the hand, arm, leg, or eye) and tremor (involuntary, somewhat rhythmic, muscle contraction and relaxation involving oscillations or twitching movements of one or more body parts). These assessments are clinically valuable in evaluation and/or diagnosis of a wide range of neurodegenerative disorders ranging from orthostatic tremor to Parkinson's' disease.

Although the objective evaluation of the FNT and similar coordination tests has proven somewhat useful in evaluating and diagnosing certain neurodegenerative disorders, there is a need for a way for an objective assessment which provides more precise results.

### SUMMARY

The present disclosure is directed to systems and methods for performing a motor skills neurological test on a patient using augmented reality which provides an objective assessment and/or quantification of the patient's performance on the test. In general, the systems and methods are implemented on a computerized augmented reality system (AR system) comprising a computer having a computer processor, memory, a storage device, and software stored on the storage device and executable to program the computer to perform operations enabling the virtual reality system. Typically, the AR system includes a wearable system such as a headset wearable by user which projects AR images into the eyes of the user, although the AR system is not required to be a wearable or to be implemented as a headset. The AR system is configured to present 3D virtual images in an AR field of view to the user which simulate accurate locations of virtual objects in a world coordinate system.

Hence, one embodiment disclosed herein is directed to a computer-implemented method of performing a motor skills neurological test using augmented reality. The method comprises displaying a virtual target to a user in an AR field of view on an AR display system at a target location in a 3D world coordinate system. For example, the virtual target may be displayed at a location representative of the nose of the user, or any other suitable location within the reach of the user. The movement of a body part of the user is tracked as the user moves the body part from a body part starting location in the 3D world coordinate system to the target location in the 3D world coordinate system. For instance, the body part may be an index finger, or other finger, of the user's hand. The starting location can be any suitable starting location, such as the location of the index finger of the user's hand outstretched to the side of the user, or in front of the user. The movement of the body part can be tracked using any suitable sensor(s), such as one or more camera(s) disposed on the headset of the AR system.

A total traveled distance of the body part of the user (e.g., a patient) in moving from the starting location to the target location in the 3D world coordinate system is determined based on tracking the movement of the body part. This is a relatively simple calculation of the length of the path of the tracked movement of the body part. Also, a linear distance between the starting location and the target location in the 3D world coordinate system is determined. This may be a simple calculation of the linear distance between the coordinates of the body part starting location in the and the coordinates of the target location in the 3D world coordinate system.

An efficiency index is then determined which represents an overall quality of movement of the body part from the starting location to the target location based on the total traveled distance and the linear distance. As one example, the efficiency index may be the ratio of the linear distance between the starting location and the target location in the 3D world coordinate system and the total traveled distance of the body part.

In another aspect of the method, the efficiency index may be proportional to the linear distance divided by the total traveled distance. In another aspect, the efficiency index may be the linear distance divided by the total traveled distance multiplied by 100.

In still another aspect, the body part may be tracked by tracking one or more keypoints representing the location of a finger of a hand of the user. For instance, one or more locations on the user's index finger may each be identified as a keypoint, and the method tracks the path of the keypoints as the user moves the index finger from the starting location to the target location.

In still another aspect, the method may further comprise detecting a user's eye tracking of the body part while tracking the movement of the body part of the user as the user moves the body part from the body part starting location to the target location. The detection of the user's eye tracking of the body part monitors the direction of the user's eye gaze during movement of the body part. This data can be used to evaluate the smoothness of the user's eye tracking during the test, and can enable more comprehensive clinical evaluation of the patient's motor skills function. In another aspect, a correlation between a proficiency of the user's eye tracking and the quality of movement of the body part from the starting location to the target location can be determined. In still another aspect, correlation data representative of the correlation between a proficiency of the user's eye tracking and the quality of movement of the body part from the starting location to the target location can be provided to the clinician. The correlation data can then be used by a clinician to further evaluate and diagnose the user's condition.

In another aspect, the test may include a series of virtual targets for the user to touch. In one aspect, the series of virtual targets may be displayed sequentially one by one as the user moves the body part to the target location of each successive virtual target. In still another feature, each virtual target may be positioned at a different target location in the 3D world coordinate system. For example, different virtual targets may include a first target representative of the location of the user's nose, a second target representative of the location of the user's right ear, a third target located in front of the user, etc.

The movement of the body part of the user is tracked as the user moves the body part from a respective starting location in the 3D world coordinate system to each respective virtual target location in the 3D world coordinate system. The total traveled distance of the body part of the user in moving the body part from the respective starting location to the respective target location, for each virtual target, in the 3D world coordinate system based on tracking the movement of the body part. A linear distance of a path comprising linear segments connecting the respective starting location and the respective target location, for each virtual target, in the 3D world coordinate system. An efficiency index which represents an overall quality of movement of the body part on the total traveled distance and the linear distance of the path is then determined. The efficiency index may be calculated similar to the efficiency index for the method using a single virtual target, such as the ratio of the linear distance of the path and the total traveled distance of the body part. In other aspects, the efficiency index may be proportional to the linear distance divided by the total traveled distance; or the linear distance divided by the total traveled distance multiplied by 100. Furthermore, the method comprising a series of virtual targets can include any one or more of the aspects and features described for the method using a single virtual target.

In another aspect of the method, the motor skills neurological test may be standardized and/or normalized for each particular user in order to ensure repeatability of the test and the reliability of the data collected and results obtained. For example, to ensure comparable results between trials for the evaluation of performance changes (i.e., improvements or degradation of a clinical condition), the method may include performing the test including a series of virtual targets in accordance with standardized clinical procedures. In addition, the test may be the exact same test with the same series of virtual targets and target locations.

In yet another aspect of the method, the tracking data, efficiency index, and/or correlation data, may be normalized to the user's anthropomorphics. For example, the test results may be normalized relative to the user's arm length and/or finger length.

In still other aspects of the method, additional metrics may be measured and analyzed into useful data for real-time feedback to the user, and for use by the clinician in evaluation, diagnosis and/or treatment of the user. For example, additional metrics may include an elapsed time to completion for the user to move the body part from the starting location in the 3D world coordinate system to the target location(s), a velocity of the movement of the body part in moving the body part from the starting location to the target location(s), and/or the spatial and temporal variability of the path of the body part in moving the body part from the starting location to the target location(s).

Another embodiment disclosed herein is directed to an AR system for performing a motor skills neurological test on a user (e.g., a patient) using augmented reality which provides an objective assessment and/or quantification of the patient's performance on the test. The AR system may be the same or similar system which performs the method embodiments described herein. Hence, in one embodiment, the AR system comprises a computer having a computer processor, memory, a storage device, and software stored on the storage device and executable to program the computer to perform operations enabling the virtual reality system. The AR system includes a display for displaying 3D virtual images (i.e., AR images) in an AR field of view to a user. The 3D virtual images simulate accurate locations of virtual objects in a world coordinate system. In one aspect, the AR system may include a wearable device, such as a headset, in which the display is housed. For example, the display may include a pair of light projectors, panel displays, or the like, and optic elements to project the 3D virtual images in the AR field of view into the eyes of the user. The AR system is configured to present 3D virtual images in an AR field of view to the user which simulate accurate locations of virtual objects in a world coordinate system. The headset also allows a degree of transparency to the real-world surrounding the user such that the AR images augment the visualization of the real-world.

The software is executable by the computer processor to program the AR system to perform a process for conducting a motor skills neurological test on a user using augmented reality. For instance, the process may include: displaying a virtual target to a user in an AR field of view on an MR display system at a target location in a 3D world coordinate system; tracking the movement of a body part of the user as the user moves the body part from a body part starting location in the 3D world coordinate system to the target location in the 3D world coordinate system; determining a total traveled distance of the body part of the user in moving from the body part starting location to the target location in the 3D world coordinate system based on tracking the movement of the body part; determining a linear distance between the body part starting location and the target location in the 3D world coordinate system; determining an efficiency index which represents an overall quality of movement of the body part from the starting location to the target location based on the total traveled distance and the linear distance.

In additional aspects, the AR system may be configured such that the process includes any combination of one or more of the aspects of the method embodiments described herein. For instance, the AR system may be configured to perform the method using a series of virtual targets, detect a user's eye tracking of the body part, normalize the efficiency index relative to an anthropomorphic characteristic of the user, etc.

Another disclosed embodiment is directed to a non-transitory computer readable medium having stored thereon a sequence of instructions which, when stored in memory and executed by a processor programs the processor to cause an AR computing system to perform a process for conducting a motor skills neurological test on a user using augmented reality according to any of the method embodiments, described herein. Accordingly, in one embodiment, the process includes: displaying a virtual target to a user in an AR field of view on an MR display system at a target location in a 3D world coordinate system; tracking the movement of a body part of the user as the user moves the body part from a body part starting location in the 3D world coordinate system to the target location in the 3D world coordinate system; determining a total traveled distance of the body part of the user in moving from the body part starting location to the target location in the 3D world coordinate system based on tracking the movement of the body part; determining a linear distance between the body part starting location and the target location in the 3D world coordinate system; determining an efficiency index which represents an overall quality of movement of the body part from the starting location to the target location based on the total traveled distance and the linear distance.

In additional aspects, the computer-readable medium includes instructions wherein the process includes any combination of one or more of the additional aspects and features of the method embodiments described herein. For instance, the process may including performing the method using a series of virtual targets, detecting a user's eye tracking of the body part, normalizing the efficiency index relative to an anthropomorphic characteristic of the user, etc. The “computer-readable medium” may be any element that may store the program associated with logic and/or information for use by or in connection with the instruction execution system, apparatus, and/or device. The computer-readable medium may be, for example, but is not limited to, an electronic, magnetic, optical, electromagnetic, infrared, or semiconductor system, apparatus or device. More specific examples (a non-exhaustive list) of the computer readable medium would include the following: a portable computer diskette (magnetic, compact flash card, secure digital, or the like), a random-access memory (RAM), a read-only memory (ROM), an erasable programmable read-only memory (EPROM, EEPROM, or Flash memory), a portable compact disc read-only memory (CDROM), digital tape, and other non-transitory media.

Additional and other objects, features, and advantages of the disclosure are described in the detail description, figures and claims.

### BRIEF DESCRIPTION OF THE DRAWINGS

The drawings illustrate the design and utility of various embodiments of the present disclosure. It should be noted that the figures are not drawn to scale and that elements of similar structures or functions are represented by like reference numerals throughout the figures. In order to better appreciate how to obtain the above-recited and other advantages and objects of various embodiments of the disclosure, a more detailed description of the present disclosures briefly described above will be rendered by reference to specific embodiments thereof, which are illustrated in the accompanying drawings. Understanding that these drawings depict only typical embodiments of the disclosure and are not therefore to be considered limiting of its scope, the disclosure will be described and explained with additional specificity and detail through the use of the accompanying drawings in which:

FIG. 1 depicts a user's view of an AR field of view on a 3D display system of an AR system, according to some embodiments.

FIGS. 2A-2B schematically depict an AR system and subsystems thereof, according to some embodiments.

FIG. 3 is a flow chart of a motor skills neurological test method performed by an AR system, according to one embodiment.

FIG. 4 is an exemplary user's view of an AR field of view according to the test method shown in FIG. 3, according to one embodiment.

FIG. 5 is an illustration depicting the use of keypoints for tracking the movement of an object, according to some embodiments.

FIG. 6 is a flow chart of a motor skills neurological test method performed by an AR system, according to another embodiment.

FIG. 7 is an illustration depicting detection of a user's eye tracking of an object, according to some embodiments.

FIG. 8 is a flow chart of a motor skills neurological test method performed by an AR system, according to another embodiment.

FIGS. 9-11 are exemplary user's views of an AR field of view according to the test method shown in FIG. 8, according to one embodiment.

### DETAILED DESCRIPTION

The following describes various embodiments of systems and methods for performing motor skills neurological tests on a patient using augmented reality in which the tests provide objective assessments and/or quantifications of the patient's performance on the test.

Various embodiments will now be described in detail with reference to the drawings, which are provided as illustrative examples of the disclosure so as to enable those skilled in the art to practice the disclosure. Notably, the figures and the examples below are not meant to limit the scope of the present disclosure. Where certain elements of the present disclosure may be partially or fully implemented using known components (or methods or processes), only those portions of such known components (or methods or processes) that are necessary for an understanding of the present disclosure will be described, and the detailed descriptions of other portions of such known components (or methods or processes) will be omitted so as not to obscure the disclosure. Further, various embodiments encompass present and future known equivalents to the components referred to herein by way of illustration.

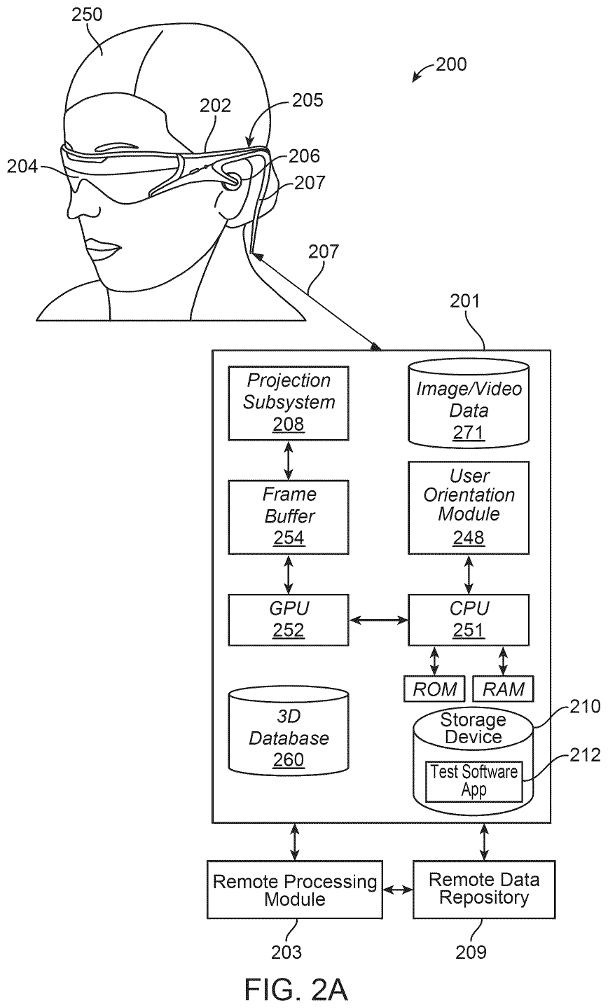

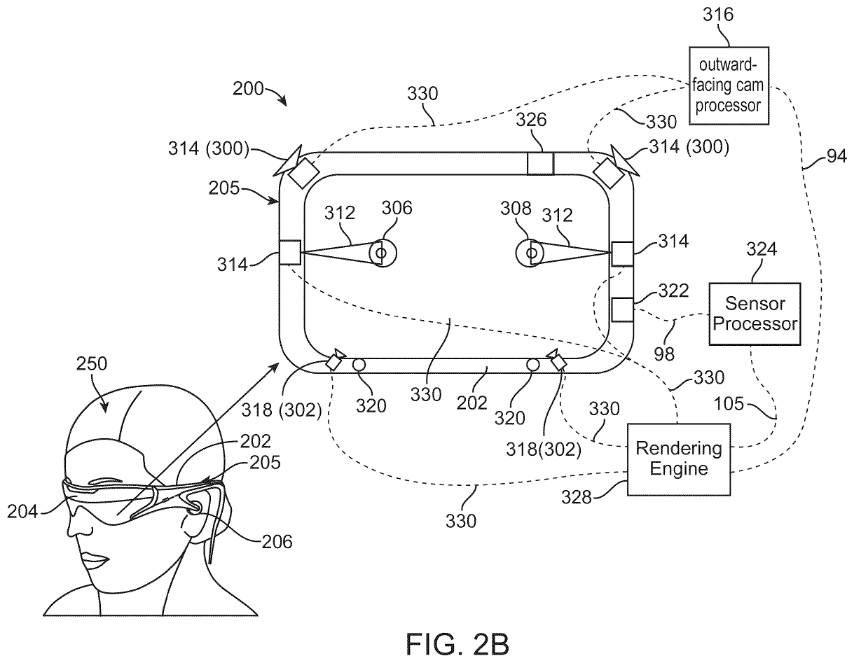

The description that follows presents an illustrative AR system 200 (see FIGS. 2A-2B) for performing motor skills neurological tests. However, it is to be understood that the embodiments also lend themselves to applications in other types of display systems (including other types of VR, AR, and/or MR systems), and therefore the embodiments are not to be limited to only the illustrative system disclosed herein.

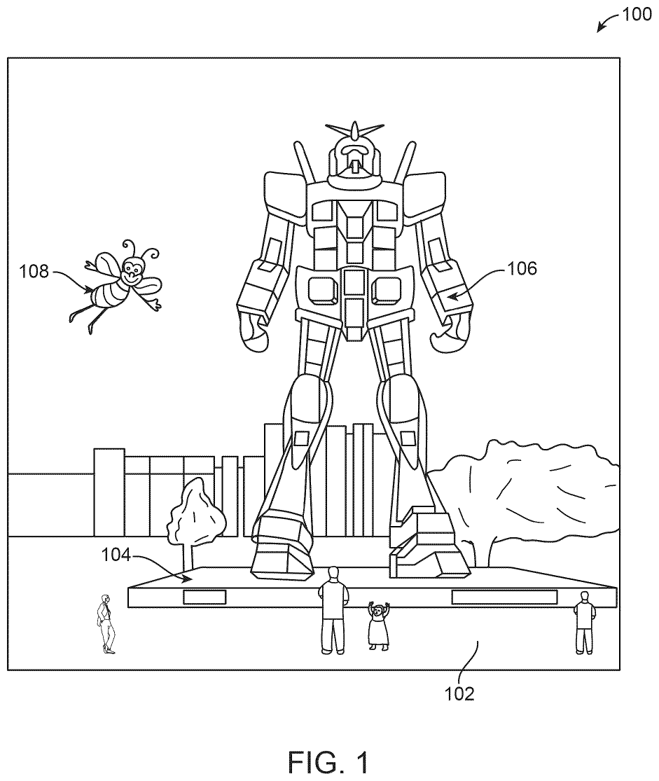

Referring to FIG. 1, AR scenarios typically include presentation of virtual content (e.g., images and sound) corresponding to virtual objects in relationship to real-world objects. For example, FIG. 1 depicts an illustration of an AR scenario with certain virtual reality objects, and certain physical, real-world objects, as viewed by a user on a 3D display system of the AR system 200 (see FIG. 2A). As shown in FIG. 1, an AR scene 100 is depicted wherein the user of AR system 200 sees a real-world, physical, park-like setting 102 featuring people, trees, buildings in the background, and a real-world, physical concrete platform 104. In addition to these items, the user of the AR system 200 also perceives that they “see” a virtual robot statue 106 standing upon the physical concrete platform 104, and a virtual cartoon-like avatar character 108 flying by which seems to be a personification of a bumblebee, even though these virtual objects 106, 108 do not exist in the real-world.

FIGS. 2A-2B illustrate an AR system 200, according to some embodiments disclosed herein. The AR system 200 is a wearable system which comprises a display-mounted headset 205 which is worn on the head of the user 250. The AR system 200 is not required to be a wearable system, but instead may include a separate display which may be a portable monitor, table-top monitor, tablet computer, smartphone or the like. However, a wearable system has the advantage of allowing the user to keep his/her hands free while using the AR system 200, and in the case of a headset, provides an immersive AR experience.

Referring to FIG. 2A, the AR system 200 includes a projection subsystem 208, providing images of virtual objects intermixed with physical objects in the AR field of view of the user 250. This approach employs one or more at least partially transparent surfaces through which an ambient environment including the physical objects can be seen and through which the AR system 200 produces images of the virtual objects. The projection subsystem 208 is housed in a control subsystem 201 operatively coupled to a display system/subsystem 204 through a link 207. The link 207 may be a wired or wireless communication link.

In typical AR applications, various virtual objects are spatially positioned relative to respective physical objects in the field of view of the user 250. The virtual objects may take any of a large variety of forms, having any variety of data, information, concept, or logical construct capable of being represented as an image. Non-limiting examples of virtual objects may include: a virtual target for a virtual text object, a virtual numeric object, a virtual alphanumeric object, a virtual tag object, a virtual field object, a virtual chart object, a virtual map object, a virtual instrumentation object, or a virtual visual representation of a physical object.

The headset 205 includes a frame structure 202 wearable by the user 250, a 3D display system 204 carried by the frame structure 202, such that the display system 204 displays rendered 3D images into the eyes 306, 308 (see FIG. 2B) of the user 250, and a speaker 206 incorporated into or connected to the display system 204. In the illustrated embodiment, the speaker 206 is carried by the frame structure 202, such that the speaker 206 is positioned adjacent (in or around) the ear canal of the user 250 (e.g., an earbud or headphone).

The display system 204 is designed to present the eyes of the user 250 with photo-based radiation patterns that can be comfortably perceived as augmentations to the ambient environment including both two-dimensional and three-dimensional content. The display system 204 presents a sequence of frames at high frequency that provides the perception of a single coherent scene. To this end, the display system 204 includes the projection subsystem 208 and a partially transparent display screen through which the projection subsystem 208 projects images. The display screen is positioned in a field of view of the user's 250 between the eyes of the user 250 and the ambient environment.

In order for the 3D display to produce a true sensation of depth, and more specifically, a simulated sensation of surface depth, it may be desirable for each point in the display's visual field to generate an accommodative response corresponding to its virtual depth. If the accommodative response to a display point does not correspond to the virtual depth of that point, as determined by the binocular depth cues of convergence and stereopsis, the human eye may experience an accommodation conflict, resulting in unstable imaging, harmful eye strain, headaches, and, in the absence of accommodation information, almost a complete lack of surface depth.

VR, AR, and MR experiences can be provided by display systems having displays in which images corresponding to a plurality of depth planes are provided to a viewer. The images may be different for each depth plane (e.g., provide slightly different presentations of a scene or object) and may be separately focused by the viewer's eyes, thereby helping to provide the user with depth cues based on the accommodation of the eye required to bring into focus different image features for the scene located on different depth plane or based on observing different image features on different depth planes being out of focus.

As one example, in order to display a 3D image in an AR field of view with objects displayed such that the user perceives the objects to be in accurate locations in a world coordinate system, in some embodiments, the projection subsystem 208 takes the form of a scan-based projection device and the display screen takes the form of a waveguide-based display into which the scanned light from the projection subsystem 208 is injected to produce, for example, images at single optical viewing distance closer than infinity (e.g., arm's length), images at multiple, discrete optical viewing distances or focal planes, and/or image layers stacked at multiple viewing distances or focal planes to represent volumetric 3D objects. These layers in the light field may be stacked closely enough together to appear continuous to the human visual subsystem (e.g., one layer is within the cone of confusion of an adjacent layer). Additionally, or alternatively, picture elements may be blended across two or more layers to increase perceived continuity of transition between layers in the light field, even if those layers are more sparsely stacked (e.g., one layer is outside the cone of confusion of an adjacent layer). The display system 204 may be monocular or binocular. The scanning assembly includes one or more light sources that produce the light beam (e.g., emits light of different colors in defined patterns). The light source may take any of a large variety of forms, for instance, a set of RGB sources (e.g., laser diodes capable of outputting red, green, and blue light) operable to respectively produce red, green, and blue coherent collimated light according to defined pixel patterns specified in respective frames of pixel information or data. Laser light provides high color saturation and is highly energy efficient. The optical coupling subsystem includes an optical waveguide input apparatus, such as for instance, one or more reflective surfaces, diffraction gratings, mirrors, dichroic mirrors, or prisms to optically couple light into the end of the display screen. The optical coupling subsystem further includes a collimation element that collimates light from the optical fiber. Optionally, the optical coupling subsystem includes an optical modulation apparatus configured for converging the light from the collimation element towards a focal point in the center of the optical waveguide input apparatus, thereby allowing the size of the optical waveguide input apparatus to be minimized. Thus, the display system 204 generates a series of synthetic image frames of pixel information that present an undistorted image of one or more virtual objects to the user. Further details describing display subsystems are provided in U.S. Utility patents application Ser. Nos. 14/212,961, entitled “Display System and Method” (Attorney Docket No. ML.20006.00), and 14/331,218, entitled “Planar Waveguide Apparatus With Diffraction Element(s) and Subsystem Employing Same” (Attorney Docket No. ML.20020.00), the contents of which are hereby expressly and fully incorporated by reference in their entirety, as though set forth in full.

The AR system 200 further includes one or more sensors mounted to the frame structure 202, some of which are described herein with respect to FIG. 2B, for detecting the position (including orientation) and movement of the head of the user 250 and/or the eye position and inter-ocular distance of the user 250. Such sensor(s) may include image capture devices (e.g., cameras in an inward-facing imaging system and/or cameras in an outward-facing imaging system), audio sensor (e.g., microphones), inertial measurement units (IMUs), accelerometers, compasses, GPS units, radio devices, gyros, and the like. For example, in one embodiment, the AR system 200 includes a head worn transducer subsystem that includes one or more inertial transducers to capture inertial measures indicative of movement of the head of the user 250. Such devices may be used to sense, measure, or collect information about the head movements of the user 250. For instance, these devices may be used to detect/measure movements, speeds, acceleration and/or positions of the head of the user 250. The position (including orientation) of the head of the user 250 is also known as a “head pose” of the user 250.

The AR system 200 of FIG. 2A includes an outward-facing imaging system 300 (see FIG. 2B) which observes the world in the environment around the user 250. The outward-facing imaging system 300 comprises one or more outward-facing cameras 314. The cameras 314 include cameras facing in all outward directions from the user 250, including the front, rear and sides of the user 250, and above and/or below the user 250. The outward-facing imaging system 300 may be employed for any number of purposes, such as detecting and tracking objects around the user, recording of images/video of the environment surrounding the user 250, and/or capturing information about the environment in which the user 250 is located, such as information indicative of distance, orientation, and/or angular position of the user 250 and objects around the user with respect to the environment around the user.

The AR system 200 may further include an inward-facing imaging system 304 (see FIG. 2B) which can track the angular position (the direction in which the eye or eyes are pointing), movement, blinking, and/or depth of focus (by detecting eye convergence) of the eyes 306, 308 of the user 250. Such eye tracking information may, for example, be discerned by projecting light at the user's eyes, 306, 308, and detecting the return or reflection of at least some of that projected light.

The augmented reality system 200 also includes a control subsystem 201 that may take any of a variety of forms. The control subsystem 201 includes a number of controllers, for instance one or more microcontrollers, microprocessors or central processing units (CPUs), digital signal processors, graphics processing units (GPUs), other integrated circuit controllers, such as application specific integrated circuits (ASICs), programmable gate arrays (PGAs), for instance field PGAs (FPGAs), and/or programmable logic controllers (PLUS). The control subsystem 201 includes a digital signal processor (DSP), one or more central processing units (CPUs) 251, one or more graphics processing units (GPUs) 252, and one or more frame buffers 254. The CPU 251 controls overall operation of the AR system 200, while the GPU 252 renders frames (i.e., translating a three-dimensional scene into a two-dimensional image) and stores these frames in the frame buffer(s) 254. While not illustrated, one or more additional integrated circuits may control the reading into and/or reading out of frames from the frame buffer(s) 254 and operation of the display system 204. Reading into and/or out of the frame buffer(s) 254 may employ dynamic addressing, for instance, where frames are over-rendered. The control subsystem 201 further includes a read only memory (ROM) and a random access memory (RAM). The control subsystem 201 further includes a three-dimensional database 260 from which the GPU 252 can access three-dimensional data of one or more scenes for rendering frames, as well as synthetic sound data associated with virtual sound sources contained within the three-dimensional scenes.

The control subsystem 201 may also include an image/video database 271 for storing the image/video and other data captured by the outward-facing imaging system 300, the inward-facing imaging system 302, and/or any other camera(s) and/or sensors of the AR system 200.

The control subsystem 201 may also include a user orientation detection module 248. The user orientation module 248 detects an instantaneous position of the head of the user 250 and may predict a position of the head of the user 250 based on position data received from the sensor(s). The user orientation module 248 also tracks the eyes of the user 250, and in particular the direction and/or distance at which the user 250 is focused based on the tracking data received from the sensor(s).

The various processing components of the AR systems 200 may be contained in a distributed subsystem. For example, the AR system 200 may include a local processing and data module (i.e., the control subsystem 201) operatively coupled, such as by a wired lead or wireless connectivity 207, to a portion of the display system 204. The local processing and data module may be mounted in a variety of configurations, such as fixedly attached to the frame structure 202, fixedly attached to a helmet or hat, embedded in headphones, removably attached to the torso of the user 250, or removably attached to the hip of the user 250 in a belt-coupling style configuration. The AR system 200 may further include a remote processing module 203 and remote data repository 209 operatively coupled, such as by a wired lead or wireless connectivity to the local processing and data module 203, such that these remote modules are operatively coupled to each other and available as resources to the local processing and data module 203. The local processing and data module 201 may comprise a power-efficient processor or controller, as well as digital memory, such as flash memory, both of which may be utilized to assist in the processing, caching, and storage of data captured from the sensors and/or acquired and/or processed using the remote processing module 203 and/or remote data repository 209, possibly for passage to the display system 204 after such processing or retrieval. The remote processing module 203 may comprise one or more relatively powerful processors or controllers configured to analyze and process data and/or image information. The remote data repository 209 may comprise a relatively large-scale digital data storage facility, which may be available through the internet or other networking configuration in a “cloud” resource configuration. In some embodiments, all data is stored and all computation is performed in the local processing and data module 201, allowing fully autonomous use from any remote modules. The couplings between the various components described above may include one or more wired interfaces or ports for providing wires or optical communications, or one or more wireless interfaces or ports, such as via RF, microwave, and IR for providing wireless communications. In some implementations, all communications may be wired, while in other implementations all communications may be wireless, with the exception of the optical fiber(s).

The AR system 200 also includes a storage device 210 for storing software applications to program the AR system 200 to perform application specific functions. The storage device 210 which may be any suitable storage device such as a disk drive, hard drive, solid state drive (SSD), tape drive, etc. The storage device 210 for storing software applications may also be any one of the other storage devices of the AR system, and is not required to be a separate, stand-alone storage device for software applications. For example, the 3D database 260, and/or image/video data 271 may be stored on the same storage device A motor skills neurological test software application 212 is stored on the storage device 210.

Turning to FIG. 2B, the AR system 200 is shown along with an enlarged schematic view of the headset 205 and various components of the headset 205. In certain implements, one or more of the components illustrated in FIG. 2B can be part of the 3D display system 204. The various components alone or in combination can collect a variety of data (such as e.g., audio or visual data) associated with the user 250 of the wearable system 200 or the user's environment. It should be appreciated that other embodiments may have additional or fewer components depending on the application for which the wearable system is used. Nevertheless, FIG. 2B illustrates one exemplary embodiment of the AR system 200 for performing motor skills neurological tests as described herein.

As shown in FIG. 2B, the AR system 200 includes the 3D display system 204. The display system 204 comprises a display lens 310 that is on the wearable frame 202. The display lens 310 may comprise one or more transparent mirrors positioned by the frame 220 in front of the user's eyes 306, 308 and may be configured to bounce projected light beams 312 comprising the AR images into the user's eyes 306, 308 and facilitate beam shaping, while also allowing for transmission of at least some light from the environment around the user 250. The wavefront of the projected light beams 312 may be bent or focused to coincide with a desired focal distance of the projected light.

Outward-facing cameras 314, which are part of the outward-facing imaging system 300, are mounted on the frame 220 and are directed outward from the user 250 to capture images (the term “image” as used herein also includes video) of the surrounding environment. The cameras 314 may be two wide-field-of-view machine vision cameras 314 (also referred to as world cameras), or any other suitable cameras or sensors. For instance, the cameras 314 may be dual capture visible light/non-visible (e.g., infrared) light cameras. Images acquired by the cameras 314 are processed by an outward-facing imaging processor 36. The outward-facing imaging processor 316 implements one or more image processing implements one or more image processing applications to analyze and extract data from the images captured by the cameras 314. The outward-facing imaging processor 316 includes an object recognition application which implements an object recognition algorithm to recognize objects within the images, including recognizing various body parts of the user, including a user's hands, fingers, arms, legs, etc. The outward-facing imaging processor 316 also includes an object tracking application which implements an object tracking algorithm which tracks the location and movement of an object registered to a world coordinate system common to the 3D virtual location of virtual objects displayed to the user 250 on the 3D display 220. In other words, the tracked location of the real objects in the real world is relative to the same world coordinate system as the virtual images in an AR field of view displayed on the 3D display 220. The outward-facing imaging processor 316 may also include a pose processing application which implements a pose detection algorithm which identifies a pose of the user 250, i.e., the location and head/body position of the user 250. The outward-facing imaging processor 316 may be implemented on any suitable hardware, such as an ASIC (application specific integrated circuit), FPGA (field programmable gate array), ARM processor (advanced reduced-instruction-set machine), or as part of the control subsystem 201. The outward-facing imaging processor 316 may be configured to calculate real or near-real time pose, location and/or tracking data using the image information output from the cameras 314.

With continued reference to FIG. 2B, the headset 205 also includes a pair of scanned-laser shaped-wavefront (e.g., for depth) light projector modules 314 having display mirrors and optics configured to project the light 312 into the user's eyes 306, 308. The headset 205 also has inward-facing cameras/sensors 318, which are part of the inward-facing imaging system 302, mounted on the interior of the frame 220 and directed at the user's eyes 306, 308. The cameras 318 may be two miniature infrared cameras 318 paired with infrared light sources 320 (such as light emitting diodes “LED”s), which are configured to track the gaze of the user's eyes 306, 308 user to support rendering of AR images, for user input (e.g., gaze activated selection of user inputs), and also to determine a correlation between a proficiency of the user's eye tracking and the quality of movement of the user's body part from a starting location to a target location, as discussed in more detail herein. The user's eye tracking data can be used to evaluate the smoothness of the user's eye tracking during the test, and can enable more comprehensive clinical evaluation of the patient's motor skills function. Furthermore, the AR system 200 is configured to determine a correlation between the proficiency of the user's eye tracking and the quality of movement of the body part from the starting location to the target location. This correlation data representative of the correlation between a proficiency of the user's eye tracking and the quality of movement of the body part from the starting location to the target location can be provided to the clinician. The correlation data can then be used by a clinician to further evaluate and diagnose the user's condition.

The AR system 200 may also have a sensor assembly 322, which may comprise an X, Y, and Z axis accelerometer capability as well as a magnetic compass and X, Y, and Z axis gyro capability, preferably providing data at a relatively high frequency, such as 200 Hz. The sensor assembly 322 may be part of the IMU described with reference to FIG. 2A.

Still referring to FIG. 2B, the AR system 200 may also include a sensor processor 324 configured to execute digital or analog processing of the data received from the gyro, compass, and/or accelerometer of the sensor assembly 322. The sensor processor 324 may be part of the local control subsystem 201 shown in FIG. 2A. The AR system 200 may also include a position system 326 such as, e.g., a GPS module 326 (global positioning system) to assist with pose and positioning analyses. In addition, the GPS 326 may further provide remotely-based (e.g., cloud-based) information about the user's environment. This information may be used for recognizing objects or information in user's environment.

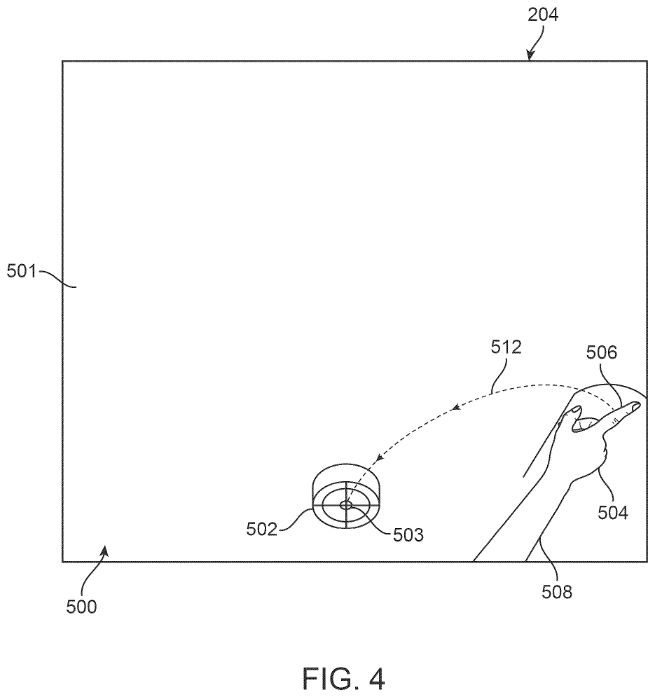

The AR system 200 may combine data acquired by the GPS 326 and a remote computing system (such as, e.g., the remote processing module 203) which can provide more information about the user's environment. As one example, the wearable system can determine the user's location based on GPS data and retrieve a world map (e.g., by communicating with a remote processing module 203) including virtual objects associated with the user's location. As another example, the wearable system 200 can monitor the environment using the cameras 16 (which may be part of the outward-facing imaging system 464 shown in FIG. 4). Based on the images acquired by the world cameras 16, the wearable system 200 can detect characters in the environment (e.g., by using the object recognition application of the outward-facing imaging processor 316). The AR system 200 can further use data acquired by the GPS 37 to interpret the characters. For example, the AR system 200 can identify a geographic region where the characters are located and identify one or more languages associated with the geographic region. The AR system 200 can accordingly interpret the characters based on the identified language(s), e.g., based on syntax, grammar, sentence structure, spelling, punctuation, etc., associated with the identified language(s). In one example, a user in Germany can perceive a traffic sign while driving down the autobahn. The AR system 200 can identify that the user is in Germany and that the text from the imaged traffic sign is likely in German based on data acquired from the GPS 37 (alone or in combination with images acquired by the cameras 314).

In some situations, the images acquired by the cameras 314 may include incomplete information of an object in a user's environment. For example, the image may include an incomplete text (e.g., a sentence, a letter, or a phrase) due to a hazy atmosphere, a blemish or error in the text, low lighting, fuzzy images, occlusion, limited FOV of the cameras 314 etc. The AR system 200 could use data acquired by the GPS 326 as a context clue in recognizing the text in image.

The AR system 200 may also comprise a rendering engine 328 which can be configured to provide rendering information that is local to the user 250 to facilitate operation of the scanners and imaging into the eyes 306, 308 of the user 250, for the user's view of the world. The rendering engine 328 may be implemented by a hardware processor (such as, e.g., a central processing unit or a graphics processing unit). In some embodiments, the rendering engine 328 is part of the control subsystem 201.

The components of the AR system are communicatively coupled to each other via one or more communication links 330. The communication links may be wired or wireless links, and may utilize any suitable communication protocol. For example, the rendering engine 328, can be operably coupled to the cameras 318 via communication link 330, and be coupled to the projection 208 (which can project light 312 into user's eyes 306, 308 via a scanned laser arrangement in a manner similar to a retinal scanning display) via the communication link 330. The rendering engine 328 can also be in communication with other processing units such as, e.g., the sensor processor 324 and the outward-facing camera processor 316 via links 330.

The cameras 318 (e.g., mini infrared cameras) may be utilized to track the eye pose to support rendering and user input. Some examples of eye poses include where the user is looking or at what depth he or she is focusing (which may be estimated with eye vergence). The GPS 326, gyros, compass, and accelerometers 322 may be utilized to provide coarse or fast pose estimates. One or more of the cameras 314 can also acquire images and pose, which in conjunction with data from an associated cloud computing resource, may be utilized to map the local environment and share user views with others.

The example components depicted in FIG. 2B are for illustration purposes only. Multiple sensors and other functional modules are shown together for ease of illustration and description. Some embodiments may include only one or a subset of these sensors or modules. Further, the locations of these components are not limited to the positions depicted in FIG. 2B. Some components may be mounted to or housed within other components, such as a belt-mounted component, a hand-held component, or a helmet component. As one example, the outward-facing camera processor 316, sensor processor 324, and/or rendering engine 328 may be positioned in a belt-pack and configured to communicate with other components of the AR system 200 via wireless communication, such as ultra-wideband, Wi-Fi, Bluetooth, etc., or via wired communication. The depicted frame 2015 may be head-mountable and wearable by the user 250. However, some components of the AR system 200 may be worn on other portions of the user's body. For example, the speaker 206 may be inserted into the ears of the user 250 to provide sound to the user 250.

Regarding the projection of light 312 into the eyes 306, 308 of the user 250, in some embodiment, the cameras 318 may be utilized to measure where the centers of a user's eyes 306, 308 are geometrically verged to, which, in general, coincides with a position of focus, or “depth of focus”, of the eyes 306, 308. A 3-dimensional surface of all points the eyes verge to can be referred to as the “horopter”. The focal distance may take on a finite number of depths, or may be infinitely varying. Light projected from the vergence distance appears to be focused to the subject eye 306, 308, while light in front of or behind the vergence distance is blurred. Examples of wearable devices and other display systems of the present disclosure are also described in U.S. Patent Publication No. 2016/0270656, which is incorporated by reference herein in its entirety.

Further spatially coherent light with a beam diameter of less than about 0.7 millimeters can be correctly resolved by the human eye regardless of where the eye focuses. Thus, to create an illusion of proper focal depth, the eye vergence may be tracked with the cameras 24, and the rendering engine 34 and projection subsystem 18 may be utilized to render all objects on or close to the horopter in focus, and all other objects at varying degrees of defocus (e.g., using intentionally-created blurring). Preferably, the system 220 renders to the user at a frame rate of about 60 frames per second or greater. As described above, preferably, the cameras 24 may be utilized for eye tracking, and software may be configured to pick up not only vergence geometry but also focus location cues to serve as user inputs. Preferably, such a display system is configured with brightness and contrast suitable for day or night use.

In some embodiments, the display system preferably has latency of less than about 20 milliseconds for visual object alignment, less than about 0.1 degree of angular alignment, and about 1 arc minute of resolution, which, without being limited by theory, is believed to be approximately the limit of the human eye. The display system 204 may be integrated with a localization system, which may involve GPS elements, optical tracking, compass, accelerometers, or other data sources, to assist with position and pose determination; localization information may be utilized to facilitate accurate rendering in the user's view of the pertinent world (e.g., such information would facilitate the glasses to know where they are with respect to the real world).

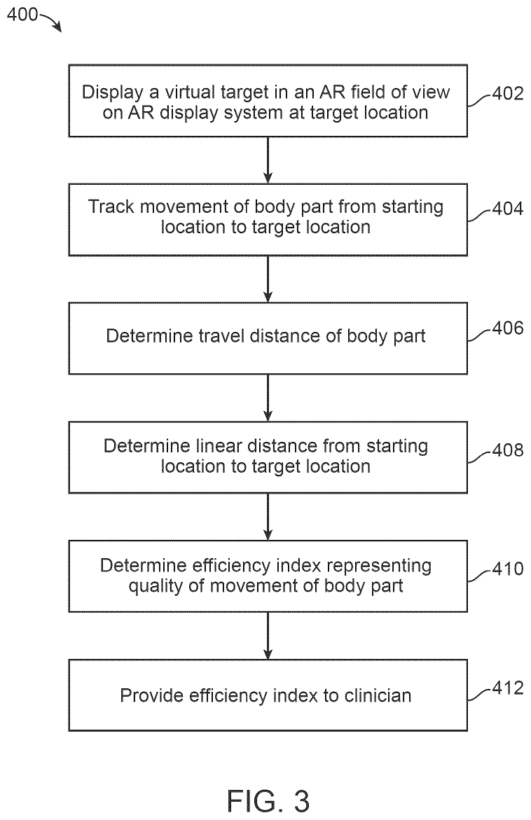

The AR system 200 is programmed by the motor skills neurological test software application 212 to perform a motor skills neurological test on a user (e.g., a patient) which provides objective assessments and/or quantifications of the user's performance on the test. FIG. 3 is a flow chart of one embodiment of a motor skills neurological test method 400 performed by the AR system 200 as programmed by the test software application 212. FIG. 4 illustrates an example of a user experience while performing the test 400 on the AR system 200. At step 402 of the test 400, as shown in FIGS. 3 and 4, the AR system 200 displays a virtual target 502 to the user 250 in an AR field of view 500 on the AR display system 204 at a target location 503 in a 3D world coordinate system of the AR field of view 500. In the example of FIG. 4, the virtual target 502 is in the form of a bullseye target. Alternatively, the virtual target 502 may be in the shape of a nose, a different shaped target, or other suitable virtual target 502. The target location 503 is positioned at a location representative of the nose of the user 250. Alternatively, the target location 503 may be at any other suitable location within the reach of the user 250. As explained herein, the AR display system 204 also allows the user 250 to view the real-world environment 501 surrounding the user 250. Hence, the user 250 may also be able to see the user's hand 504, pointer finger 506, and part of the arm 508. In the example tests described herein, the body part used in the test (e.g., test 400) is the user's pointer finger 506. Any other suitable body part of the user 250 may be used for the test, such as a different finger of the user 250, the user's arm 508, the user' foot, etc.

Depending on the starting location of the pointer finger 506, the user's hand 504, pointer finger 506, and part of the arm 508 may, or may not be visible in the AR field of view 500 at the start of the test 400, but would become visible in the AR field of view 500 as the user 250 moves the pointer finger 506 toward the virtual target 502. For example, the starting location of the pointer finger 506 may be the position of the pointer finger 506 with the user's arm 508 outstretched directly to the side of the user 250. If that is the case, the user's hand 504, pointer finger 506, and part of the arm 508 may not be visible in the AR field of view 500. As the user 250 moves the pointer finger 506 from the starting location (e.g., outstretched to the side of the user 250) to the target location 503 of the virtual target 502, at step 404, the AR system 200 tracks the movement of the pointer finger 506. The AR system 200 tracks the movement of the pointer finger 506 using the outward-facing camera system 300, including the cameras 314 and the outward-facing camera processor 316. The cameras 314 obtain images of the user's hand 504, pointer finger 506, and part of the arm 508, and communicate the images to the outward-facing imaging processor 316. The outward-facing camera processor 316 processes the images, including the user's hand 504, pointer finger 506, and part of the arm 508 (or other relevant body part of the user 250) using the object recognition application, and tracks the movement of the pointer finger 506 in the 3D world coordinate system using the object tracking application.

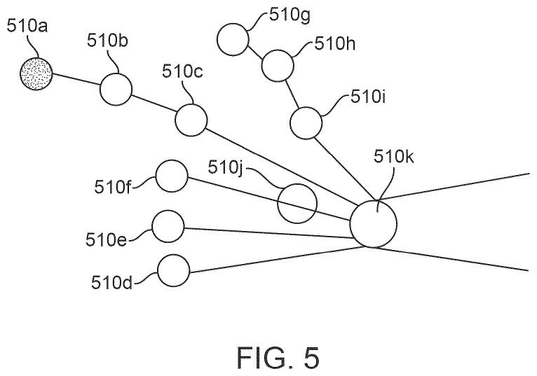

As illustrated in FIG. 5, the outward-facing camera processor 316 may track user's hand 504, pointer finger 506, and/or part of the arm 508 (or other relevant body part) using a 3D keypoint algorithm. The 3D keypoint algorithm identifies one or more keypoints 510, such a first keypoint 510a representing the tip of the user's pointer finger 506, keypoints 510b-510c representing the knuckles of the pointer finger 506, keypoints 510d-510f representing the knuckles of the user's other fingers, keypoints 510g-510i representing the user's thumb, keypoint 510j representing the palm of the user's hand 504, and keypoint 510k representing the user's wrist. The location of a finger of a hand of the user. For instance, one or more locations on the user's index finger may each be identified as a keypoint, and the method tracks the path of the keypoints as the user moves the index finger from the starting location to the target location. The outward-facing camera processor 316 may use the object recognition application to identify each of these keypoints on the user's body, and then uses the 3D keypoint algorithm to track each of the keypoints 510 as the user moves the pointer finger 506 from the starting location to the target location 503. A suitable 3D keypoint algorithm is described in U.S. Patent Application Publication No. 2021/0302587, the contents of which is incorporated by reference in its entirety.

The path 512 shows one example of the user's pointer finger 506 in moving from the starting location to the target location 503 of the virtual target 502. At step 406, the AR system 200 (e.g., the outward-facing camera processor 316) determines a total traveled distance of the pointer finger 506 in moving from the starting location to the target location 503 in the 3D world coordinate system based on tracking the movement of the pointer finger 506, i.e., the AR system 200 calculates the length of the path 512. At step 408, the AR system 200 (e.g., the outward-facing camera processor 316), determines the linear distance between the starting location and the target location 503 in the 3D world coordinate system. This may be a simple calculation of the linear distance between the coordinates of the starting location in the and the coordinates of the target location 503 in the 3D world coordinate system.

At step 410, the AR system 200 determines an efficiency index which represents an overall quality of movement of the body part from the starting location to the target location 203 based on the total traveled distance and the linear distance. In one embodiment, the efficiency index (EI) may be the ratio of the linear distance (in millimeters (mm), for example) between the starting location and the target location 203 in the 3D world coordinate system and the total traveled distance of the pointer finger 506 (in mm, for example). In another embodiment, the EI may be the ratio of the linear distance (in millimeters (mm), for example) between the starting location and the target location 203 in the 3D world coordinate system and the total traveled distance of the pointer finger 506 (in mm, for example) multiplied by 100, according to the formula set forth below:

$$EI = \frac{linear distance ( mm )}{total traveled distance ( mm )} * 100$$

At step 412 of the test 400, the AR system 200 provides the determined efficiency index to a clinician. The AR system 200 may provide the efficiency index by any suitable method, such as displaying the efficiency index on a computer display station, transmitting the efficiency index to the clinician, etc.

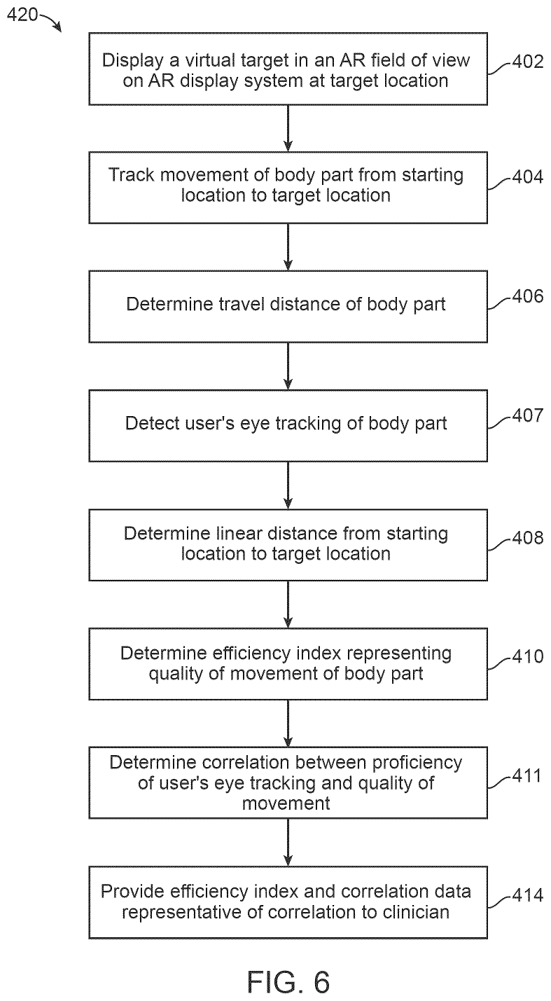

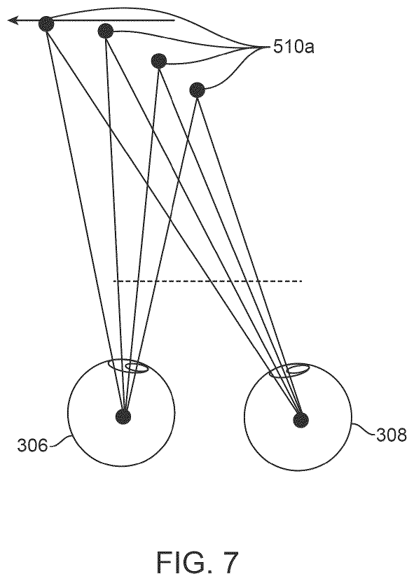

Turning to FIG. 6, a flow chart for another embodiment of a motor skills neurological test 420 performed by the AR system 200 as programmed by the test software application 212 is illustrated. The test 410 is similar to the test 400, and the steps having the same reference numbers in FIG. 3 are the same steps as for the test 420 in FIG. 6, and the description above for test 400 applies equally to test 420. The test 420 differs from test 400 in that test 420 also includes tracking the user's eyes 306, 308. Accordingly, at step 407 of test 420, the AR system 200 detects the user's eye tracking of the user's pointer finger 506 as the user 250 moves the pointer finger 506 from the starting location to the target location 503 of the virtual target 502. The AR system 200 detects the user's eye tracking using the inward-facing camera system 302 including the cameras 318, the sensor processor 324 and/or the rendering engine 328. The inward-facing camera system 302 detects the user's eye tracking of the pointer finger 506 and monitors the direction of the user's eye gaze during movement of the pointer finger 506 from the starting location to the target location 503. FIG. 7 illustrates an example of relatively smooth eye tracking by the user's eyes 306, 308 in tracking the tip of the user's pointer finger 506 as represented by keypoint 510a. As the pointer finger 506 moves from right to left, the eye gaze direction lines show the eyes 506, 508 smoothly tracking the pointer finger 506.

At step 411, the AR system 200 determines a correlation between a proficiency of the user's eye tracking and the quality of movement of the pointer finger 506 from the starting location to the target location 503. At step 414, the efficiency index and correlation data representative of the correlation between a proficiency of the user's eye tracking and the quality of movement of the pointer finger 506 are provided to the clinician. The clinician can then use this information evaluate and/or diagnose the user's condition.

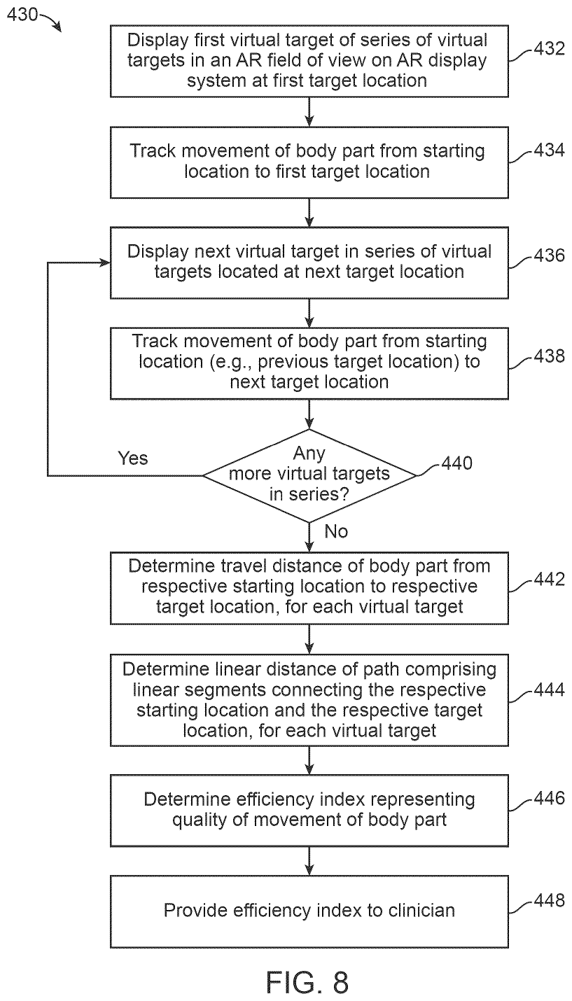

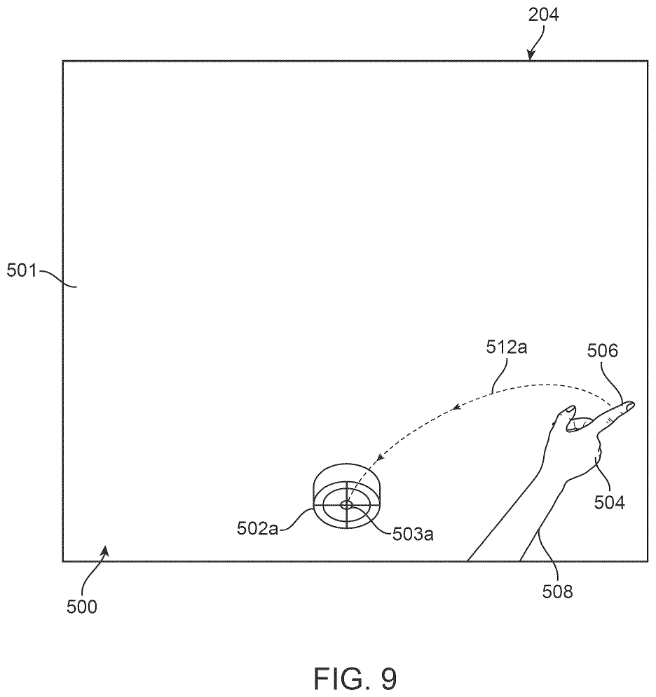

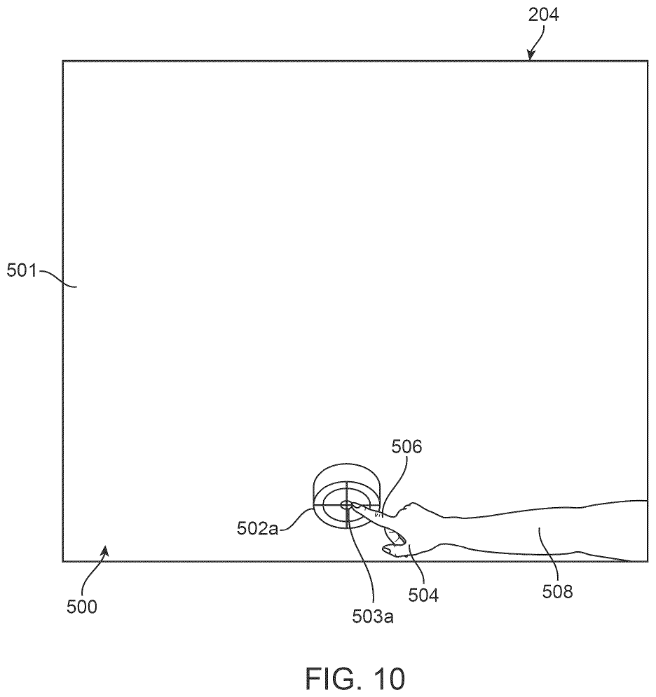

Turning now to FIGS. 8-10, a flow chart for still another embodiment of a motor skills neurological test 430 performed by the AR system 200 as programmed by the test software application 212 is illustrated. The test 430 is similar to the test 400, except that the test 430 utilizes a series of virtual targets 502 for the user 250 to move the pointer finger 506 to touch each of the virtual targets 502. As shown in FIGS. 8 and 9, at step 432, the AR system 200 displays a first virtual target 502a at a first target location 503a in a 3D world coordinate system of the AR field of view 500. At step 434, the AR system 200 tracks the movement of the pointer finger 506 as the user 250 moves the pointer finger 506 from the starting location (e.g., outstretched to the side of the user 250) to the first target location 503a of the first virtual target 502a. The AR system 200 tracks the pointer finger 506 during the test 430 in the same manner as for step 404 described above. FIG. 10 shows the pointer finger 506 moved to the first target location 503a of the first virtual target 502a.

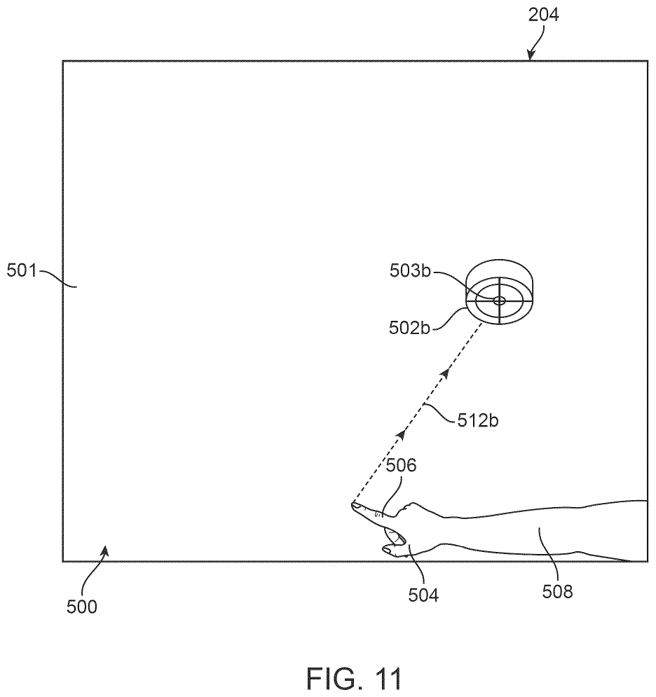

Once the user 250 has successfully moved the pointer finger 506 to the first virtual target 502a, and the AR system 200 has tracked the movement of the pointer finger 506, at step 436, the AR system 200 displays the next virtual target 502b located at a next target location 503b in the world coordinate system, as shown in FIG. 11. The next target location 503b is different than the previous (in this case, first) target location 503a. The AR system 200 may also stop displaying the first virtual target 502a, as depicted in FIG. 11.

At step 438, the AR system 200 tracks the movement of the pointer finger 506 as the user 250 moves the pointer finger 506 from a respective next starting location to the next target location 503b of the next virtual target 502b. The next starting location may be the previous target location 503, or the original starting location, such as if the user 250 is asked to re-position the pointer finger 506 back to the original starting location, e.g., the location of the pointer finger 506 with the user's arm 508 outstretched to the side of the user 250).

As one example, the series of virtual targets may include targets 502x positioned at representative locations of the user's body parts, such as a first target 502a representative of the location of the user's nose, a second target 502b representative of the location of the user's right ear, a third target 502c located in front of the user, etc.

At step 440, the AR system 200 determines if there are any more virtual targets 502 in the series of virtual targets in the current test 430. If yes, then the AR system 200 repeats steps 436-440. When there are no more virtual targets in the current test 430, the test 430 proceeds to step 442.

At step 442, the AR system 200 determines the total traveled distance of the pointer finger 506 in moving the pointer finger the respective starting location to the respective target location 503x, for each virtual target 502x, in the 3D world coordinate system based on tracking the movement of the pointer finger 506. The AR system 200 may determine the totaled travel distance for each path 512x as each virtual target 502x is reached by the user's pointer finger 506, or it may determine the total traveled distance only after the user has successfully touched all of the targets 502x in the series of virtual targets for the current test 430.

At step 448, the AR system 200 determines the linear distance of a path comprising linear segments connecting the respective starting location and the respective target location 503x, for each virtual target 502x, in the 3D world coordinate system. At step 450, the AR system determines an efficiency index which represents an overall quality of movement of the body part on the total traveled distance and the linear distance of the path is then determined. Step 450 may be performed in the same or similar manner as in step 410 for test 400, as described herein.

The test 430 may also include the aspects of the detection of the user's eye tracking of the pointer finger 506, as described for test 420.

In addition, all of the embodiments of test described herein, including test 400, 420 and 430 may be standardized and/or normalized for each particular user 250 in order to ensure repeatability of the test and the reliability of the data collected and results obtained. In one embodiment, to ensure comparable results between trials for the evaluation of performance changes (i.e., improvements or degradation of a clinical condition), the tests 400, 420 and/or 430 may include performing the test in accordance with standardized clinical procedures. In addition, each trial of a test 400, 420 and/or 430 for a particular user, may be the exact same test with the same series of virtual targets 502 and target locations 503.

Furthermore, the tracking data, efficiency index, and/or correlation data for any of the test 400, 420 and/or 430, may be normalized to the anthropomorphics of the user 250. In one embodiment, the test results may be normalized relative to the length of the user's arm 508, and/or the length of the user's finger 506, and/or the size of the user's hand, etc. The normalization may utilize a simple anthropomorphic calculation based on commonly published arm-length to standing height ratios. This value can be variable and is based on a percentage of an individual's height.

Moreover, the AR system 200 may be further configured to determine additional metrics and analyze such metrics into useful data for real-time feedback to the user 250, and for use by the clinician in evaluation, diagnosis and/or treatment of the user 250. In some embodiments, the additional metrics may include an elapsed time to completion for the user to move the body part from the starting location in the 3D world coordinate system to the target location(s) 503, a velocity of the movement of the pointer finger 506 (or other relevant body part) in moving the pointer finger 506 from respective starting location(s) to a respective target location(s) 503, and/or the spatial and temporal variability of the path 512 of the pointer finger 506 in moving the pointer finger 506 from the respective starting location(s) to the respective target location(s).

The disclosure includes methods that may be performed using the disclosed systems and devices. The methods may comprise the act of providing such suitable systems and device. Such provision may be performed by the user. In other words, the “providing” act merely requires the user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the disclosure, together with details regarding material selection and manufacture have been set forth above. As for other details of the present disclosure, these may be appreciated in connection with the above-referenced patents and publications as well as generally known or appreciated by those with skill in the art. The same may hold true with respect to method-based aspects of the disclosure in terms of additional acts as commonly or logically employed.

In addition, though the disclosure has been described in reference to several examples optionally incorporating various features, the disclosure is not to be limited to that which is described or indicated as contemplated with respect to each variation of the disclosure. Various changes may be made to the disclosure described and equivalents (whether recited herein or not included for the sake of some brevity) may be substituted without departing from the true spirit and scope of the disclosure. In addition, where a range of values is provided, it is understood that every intervening value, between the upper and lower limit of that range and any other stated or intervening value in that stated range, is encompassed within the disclosure.

Also, it is contemplated that any feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present disclosure is not to be limited to the examples provided and/or the subject specification, but rather only by the scope of claim language associated with this disclosure.

In the foregoing specification, the disclosure has been described with reference to specific embodiments thereof. It will, however, be evident that various modifications and changes may be made thereto without departing from the broader spirit and scope of the disclosure. For example, the above-described process flows are described with reference to a particular ordering of process actions. However, the ordering of many of the described process actions may be changed without affecting the scope or operation of the disclosure. The specification and drawings are, accordingly, to be regarded in an illustrative rather than restrictive sense.

1. A computer-implemented method of performing a motor skills neurological test using augmented reality, comprising:
displaying a virtual target to a user in an MR field of view on an MR display system at a target location in a 3D world coordinate system;
tracking movement of a body part of the user as the user moves the body part from a body part starting location in the 3D world coordinate system to the target location in the 3D world coordinate system;
determining the total traveled distance of the body part of the user in moving the body part from each respective starting location to the respective target location in the 3D world coordinate system based on tracking the movement of the body part;
determining a linear distance between the body part starting location and the target location in the 3D world coordinate system;
determining an efficiency index which represents an overall quality of movement of the body part from the starting location to the target location based on the total traveled distance and the linear distance.

2. The method of claim 1, wherein the efficiency index is proportional to the linear distance divided by the total traveled distance.

3. The method of claim 2, wherein the efficiency index equals (the linear distance divided by the total traveled distance) multiplied by 100.

4. The method of claim 1, wherein the body part is a finger of the user.

5. The method of claim 4, wherein the body part is tracked by tracking one or more keypoints representing the location of a finger of a hand of the user.

6. The method of claim 5, wherein the target location is representative of a nose of the user.

7. The method of claim 1, further comprising:
detecting a user's eye tracking of the body part while tracking the movement of the body part of the user as the user moves the body part from the body part starting location to the target location; and
determining a correlation between a proficiency of the user's eye tracking and the quality of movement of the body part from the starting location to the target location.

8. The method of claim 7, further comprising:
providing correlation data representative of the correlation between a proficiency of the user's eye tracking and the quality of movement of the body part from the starting location to the target location.

9. The method of claim 1, wherein the determination of the efficiency index includes normalizing a calculation of the efficiency index relative to an anthropomorphic characteristic of the user.

10. The method of claim 9, wherein the anthropomorphic characteristic is a length of the user's arm.

11. The method of claim 1, further comprising:
determining one or more of the following metrics: (1) an elapsed time to completion for the user to move the body part from the starting location to the target location; (2) a velocity of the movement of the body part in moving the body part from the starting location to the target location; (3) a spatial variability of a path of the body part in moving from the starting location to the target location; and (4) a temporal variability a path of the body part in moving from the starting location to the target location; and
providing an indication for each of the one or more metrics which is representative of the respective metric.

12.-22. (canceled)

23. An AR system for performing a motor skills neurological test using augmented reality, comprising:
a computer system having at least one computer processor, memory, a storage device, a test software application stored on the storage device; and
an AR display system for displaying virtual images in an AR field of view generated by the computer system to a user,
wherein the test software application is executable by the computer processor to program the AR system to perform a process comprising:
displaying a virtual target to a user in an MR field of view on an MR display system at a target location in a 3D world coordinate system;
tracking movement of a body part of the user as the user moves the body part from a body part starting location in the 3D world coordinate system to the target location in the 3D world coordinate system;
determining the total traveled distance of the body part of the user in moving the body part from each respective starting location to the respective target location in the 3D world coordinate system based on tracking the movement of the body part;
determining a linear distance between the body part starting location and the target location in the 3D world coordinate system;
determining an efficiency index which represents an overall quality of movement of the body part from the starting location to the target location based on the total traveled distance and the linear distance.

24. The AR system of claim 23, wherein the efficiency index is proportional to the linear distance divided by the total traveled distance.

25. The AR system of claim 24, wherein the efficiency index equals (the linear distance divided by the total traveled distance) multiplied by 100.

26. The AR system of claim 23, wherein the body part is a finger of the user, and
wherein the body part is tracked by tracking one or more keypoints representing the location of a finger of a hand of the user.

27. (canceled)

28. (canceled)

29. The AR system of claim 23, wherein the process further comprises:
detecting a user's eye tracking of the body part while tracking the movement of the body part of the user as the user moves the body part from the body part starting location to the target location; and
determining a correlation between a proficiency of the user's eye tracking and the quality of movement of the body part from the starting location to the target location.

30. The AR system of claim 29, wherein the process further comprises:
providing correlation data representative of the correlation between a proficiency of the user's eye tracking and the quality of movement of the body part from the starting location to the target location.

31. The AR system of claim 23, wherein the determination of the efficiency index includes normalizing a calculation of the efficiency index relative to an anthropomorphic characteristic of the user.

32. (canceled)

33. The AR system of claim 23, wherein the process further comprises:
determining one or more of the following metrics: (1) an elapsed time to completion for the user to move the body part from the starting location to the target location; (2) a velocity of the movement of the body part in moving the body part from the starting location to the target location; (3) a spatial variability of a path of the body part in moving from the starting location to the target location; and (4) a temporal variability a path of the body part in moving from the starting location to the target location; and
providing an indication for each of the one or more metrics which is representative of the respective metric.

34. A non-transitory computer-readable medium having software instructions stored thereon, the software instructions executable by a computer processor to cause the processor to cause an AR computing system to perform a process comprising:
displaying a virtual target to a user in an MR field of view on an MR display system at a target location in a 3D world coordinate system;
tracking movement of a body part of the user as the user moves the body part from a body part starting location in the 3D world coordinate system to the target location in the 3D world coordinate system;
determining the total traveled distance of the body part of the user in moving the body part from each respective starting location to the respective target location in the 3D world coordinate system based on tracking the movement of the body part;
determining a linear distance between the body part starting location and the target location in the 3D world coordinate system;
determining an efficiency index which represents an overall quality of movement of the body part from the starting location to the target location based on the total traveled distance and the linear distance.

35.-44. (canceled)
